(19) United States
(12) Plant Patent Application Publication
(10) Pub. No.: US 2015/0195973 P1
(43) Pub. Date: Jul. 9, 2015

(54) Citrus rootstock named 'UFR-2'

(50) ;

(71) Applicant: Florida Foundation Seed Producers, Inc., Marianna, FL (US)

(72) Inventor: Jude W. Grosser, Winter Haven, FL (US)

(21) Appl. No.: 14/544,423

(22) Filed: Jan. 5, 2015

Related U.S. Application Data
(60) Provisional application No. 61/923,587, filed on Jan. 3, 2014.

(51) Int. Cl. A01H 5/00

(52) U.S. Cl. PLT/201

(57) ABSTRACT

‘UFR-2’ is a new and distinct allotetraploid citrus rootstock for tree size control and improved disease resistance. ‘UFR-2’ has shown a positive reaction to the Huanglongbing disease (HLB, or citrus greening disease) in multiple experimental field trials. Scion trees grafted on this rootstock show a reduced frequency of infection and reduced disease symptoms once infected as compared to commercial diploid rootstocks.

### CROSS REFERENCE TO RELATED APPLICATIONS

This application claims the benefit of U.S. Provisional Application No. 61/923,587, filed Jan. 3, 2014, herein incorporated by reference in its entirety.

### BACKGROUND OF THE INVENTION

### Latin Name of the Genus and Species of the Plant Claimed

Citrus reticulata

### Variety Denomination

‘UFR-2’

The present invention relates to a new and distinct variety of citrus rootstock named ‘UFR-2’. The Plant Improvement Team in Lake Alfred, Fla. has pioneered the development and testing of allotetraploid citrus rootstocks. ‘UFR-2’ (identified as Orange #4 in field trials) is an allotetraploid zygotic hybrid derived from a conventional cross of two somatic hybrids previously produced by protoplast fusion. The somatic hybrid seed parent is ‘Nova’ mandarin hybrid+Hirado Buntan pummelo (zygotic seedling), and the somatic hybrid pollen parent is Cleopatra mandarin+Argentine trifoliate orange.

### BRIEF SUMMARY OF THE INVENTION

‘UFR-2’ was selected as a potential citrus rootstock on the basis of its positive reaction to Huanglongbing disease (HLB, or citrus greening disease) in multiple experimental field trials. Scion trees grafted onto this rootstock showed a reduced frequency of infection and reduced disease symptoms once infected when compared to other commercial diploid rootstocks. ‘UFR-2’ is seedy and highly polyembryonic, making it amenable to standard nursery propagation practices for uniform liner production. Long-term performance of trees on this rootstock selection is unknown. Yield and fruit quality data is limited, but 3 years of data from young trees (the St. Helena project, see Table 1) indicates good productivity and fruit quality. Tolerance to citrus blight is also unknown, but under investigation.

TABLE 1
Rootstock Data from 5-year old trees in the St. Helena trial - Dundee, FL

| | | | | | Yield Boxes/Tree | | | Cumulative |
|---|---|---|---|---|---|---|---|---|
| | | | Lbs Solids/Box | | 2011 | 2012 | 2013 | Yield |
| Scion | Type | Rootstock | 2012 | 2013 | (35 mo.) | (47 mo.) | (59 mo.) | (Boxes) |
| VALQUARIUS | Som. Hyb. | ‘UFR-6’ | 5.64 | 5.43 | 0.5 | 0.78 | 1.94 | 3.22 |
| VERNIA | Som. Hyb. | ‘UFR-6’ | 5.67 | 6.01 | 0.4 | 0.63 | 1.41 | 2.44 |
| VALQUARIUS | Tetrazyg | ‘UFR-1’ | 5.5 | 4.87 | NS | 0.72 | 2.23 | 2.95 |
| VERNIA | Tetrazyg | ‘UFR-1’ | 5.61 | 6.28 | 0.31 | 0.67 | 1.33 | 2.31 |
| VERNIA | Tetrazyg | ‘UFR-2’ | 5.47 | 5.93 | 0.35 | 0.25 | 1.38 | 1.98 |
| VALQUARIUS | Tetrazyg | ‘UFR-2’ | 4.57 | 5.37 | NS | 0.75 | 1.73 | 2.48 |
| VALQUARIUS | Tetrazyg | ‘UFR-3’ | 4.84 | 5.05 | NS | 0.81 | 1.97 | 2.78 |
| VERNIA | Tetrazyg | ‘UFR-3’ | 5.46 | 5.82 | 0.37 | 0.38 | 1.82 | 2.57 |
| VERNIA | Tetrazyg | ‘UFR-4’ | 5.79 | 6.07 | 0.54 | 0.71 | 1.73 | 2.98 |
| VALQUARIUS | Tetrazyg | ‘UFR-4’ | 4.65 | 5.07 | NS | 0.65 | 1.59 | 2.64 |
| VALQUARIUS | Tetrazyg | ‘UFR-5’ | 5.76 | 5.72 | 0.33 | 0.56 | 1.80 | 2.69 |
| VERNIA | Tetrazyg | ‘UFR-5’ | 5.89 | 5.34 | 0.42 | 0.25 | 1.93 | 2.60 |
| VALQUARIUS | Diploid | FG 1731 | 5.83 | 6.81 | NS | 0.68 | 2.20 | 2.88 |
| VALQUARIUS | Diploid | FG 1733 | 5.12 | 5.63 | NS | 0.67 | 2.77 | 3.44 |
| VERNIA | Diploid | SWINGLE* | 5.11 | 5.79 | 0.33 | 0.85 | 1.08 | 2.26 |
| VALQUARIUS | Diploid | SWINGLE* | NS | 5.61 | NS | NS | 1.50 | 1.50 |
| VERNIA | Diploid | CLEO* | 4.79 | 5.51 | NS | 0.50 | 0.83 | 1.33 |
| VALQUARIUS | Diploid | CLEO* | NS | 5.21 | NS | NS | 1.7 | 1.7 |
| VERNIA | Diploid | R. LEMON* | 3.67 | na | NS | 0.78 | na | 0.78 |
| VALQUARIUS | Diploid | VOLK* | NS | 4.12 | NS | NS | 2.58 | 2.58 |
| VERNIA | Diploid | VOLK* | 3.6 | 4.73 | 0.4 | 1.13 | 0.83 | 2.36 |
| VALQUARIUS | Diploid | KUHARSKE* | NS | 5.75 | NS | NS | 2.2 | 2.2 |
| VERNIA | Diploid | KUHARSKE* | 4.34 | 5.83 | 0.15 | 0.75 | 1.08 | 1.98 |

NS—not significant fruit;
na—data not available;
*control commercial rootstock
One box contains approximately 90 lbs. fruit.

### BRIEF DESCRIPTION OF THE DRAWINGS

‘UFR-2’ is illustrated by the accompanying photographs, which show the tree's form, foliage, and fruit. The colors shown are as true as can be reasonably obtained by conventional photographic procedures. The photographs are of a tree approximately 6 years old. All figures were taken in the fall of 2013.

FIG. 1.—Shows a close-up of the nearly mature fruits with the rind and cross-sectional view of the fruit when cut in the center.

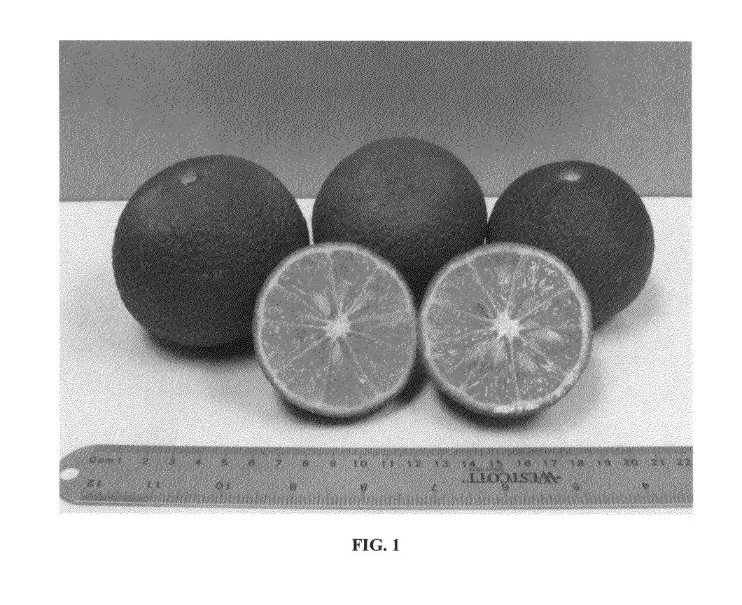

FIG. 2.—Shows the overall mature plant growth habit in October 2013.

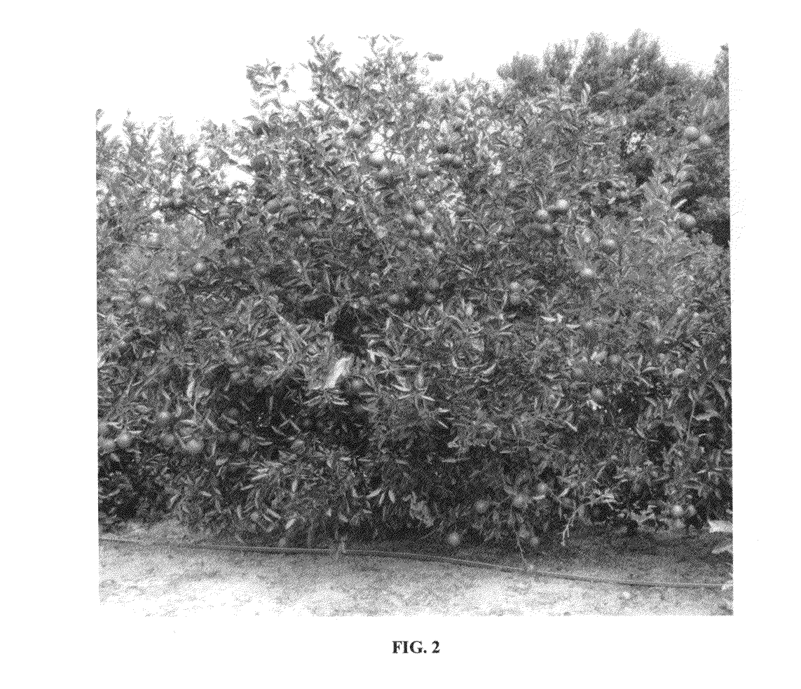

FIG. 3.—Shows nearly mature fruits hanging on the tree.

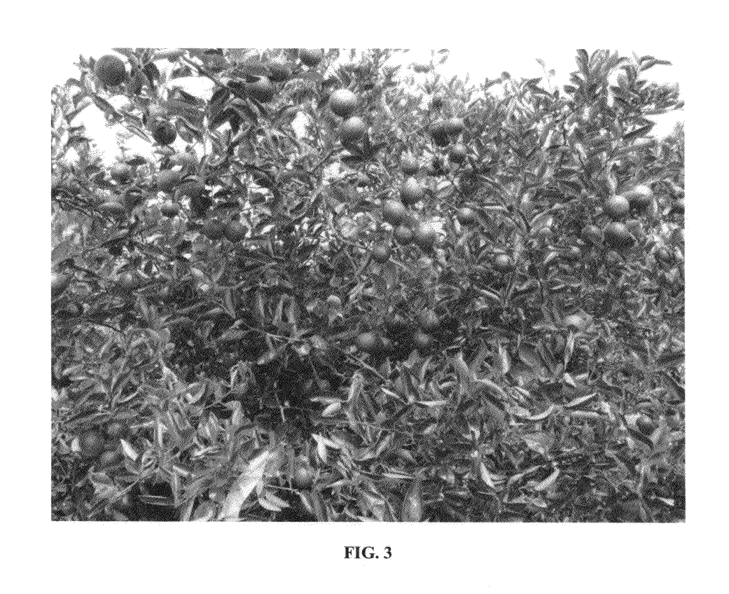

FIG. 4.—Shows a close-up of leaves and nearly mature fruits.

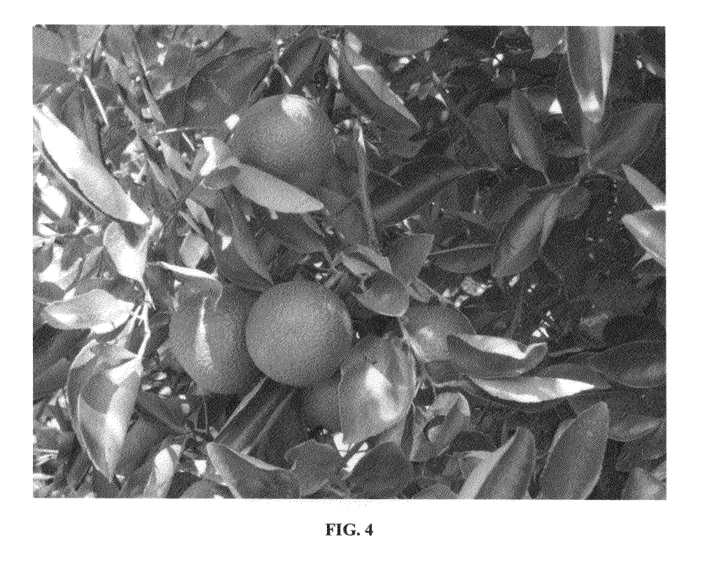

FIG. 5.—Shows a close-up of nearly mature fruits.

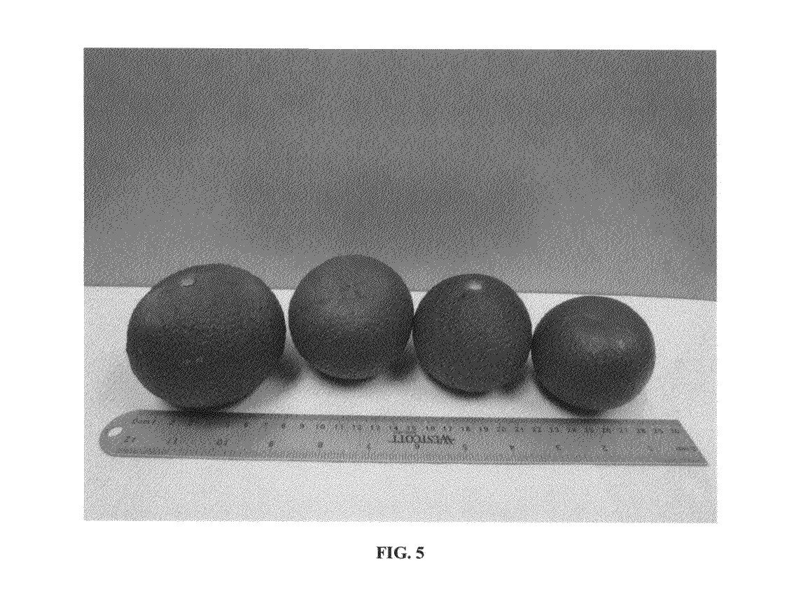

FIG. 6.—Shows a close-up of seeds from nearly mature fruit.

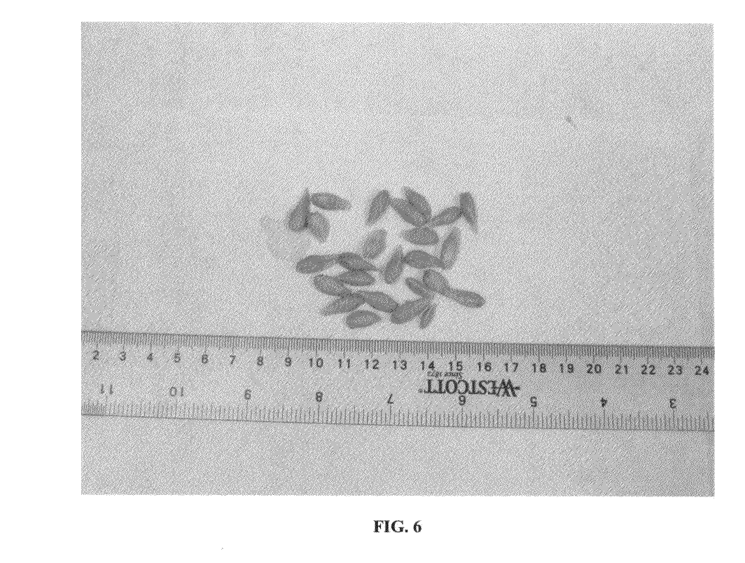

### DETAILED BOTANICAL DESCRIPTION

The following detailed description sets forth the distinctive characteristics of ‘UFR-2’. The colors (except those in common terms) are described from R.H.S. Colour Chart published by The Royal Horticultural Society in London (second edition), in association with the Flower Council of Holland.

### PHENOTYPIC DESCRIPTION OF CITRUS RETICULATA ‘UFR-2’

Classification:
   Botanical.—[Citrus reticulata hybrid (Clementine mandarin×Orlando tangelo) Citrus reticulata×Citrus paradisi)+Citrus grandis]×[Citrus reticulata+Poncirus trifoliata (trifoliate orange)].
   Common name.—Complex allotetraploid ‘tetrazyg’ rootstock hybrid.
Parentage:
   Female parent.—‘Nova mandarin+Hirado Buntan pummelo (zygotic seedling) somatic hybrid (unpatented).
   Male parent.—Cleopatra mandarin+Argentine trifoliate orange; somatic hybrid (unpatented).
Tree:
   Ploidy.—Tetraploid
   Size.—Medium.
   Height.—3.2 meters.
   Tree spread.—2.6 to 3.0 meters.
   Vigor.—Vigorous.
   Density.—Canopies are quite dense.
   Form.—The tree has a round shape with mostly lateral branches growing toward low to medium angles. Branch with fruit exhibited drooping.
   Growth habit.—Mostly lateral growth with upright growth on the top tree canopy.
Trunk:
   Trunk diameter.—12 cm in diameter at 30 cm above the ground on a 6-year-old tree.
   Trunk texture.—Smooth.
   Trunk bark color.—RHS 198A (greyed-green); irregularly striated with RHS143A (green).
Branches:
   Crotch angle.—First crotch forms 15- to 20-degree angle, middle crotch forms an 80-degree angle.
   Branch length.—Branch reaches 3.0 meters from the first crotch to the tip of the branch.
   Branch texture.—Relatively rough with small thorns or spines Branch color (shoots from previous flush, hardened and 4 to 5 mm in diameter): RHS N137A (green).
Leaves:
   Size (lamina average).—Length: 89.9 mm Width: 55.5 mm L/W ratio: 1.6.
   Thickness.—Thicker than average diploid citrus rootstock hybrids
   Type.—Mostly simple leaf types, occasionally with bifoliate and trifoliate types of leaves appearing within the same tree.
   Shape.—Elliptical.
   Apex.—Retuse.
   Base.—Acute to sub-obtuse.
   Margin.—Entire and slightly undulate.
   Surface.—Upper surface: Glabrous Lower surface: Medium veins that are pinnately netted.
   Color.—Upper surface (adaxial): RHS N137A (green) Lower surface (abaxial): RHS 137C (green).
   Petiole.—Shape: Brevipetiolate (shorter than leaf lamina); junction between petiole and lamina is articulate. Width (petiole wing): Narrow Shape (petiole wing): Obovate Length: 16.9 to 17 mm Width: 3.9 to 4.0 mm Color: RHS N137 (green).
Flowers and flower buds:
   Type.—Hermaphrodite.
   Bearing.—Flower grown from leaf axillaries and leaf terminals singly and in small clusters; most single flowers grow from leaf axillaries.
   Flower bud size.—Shape: Initial visible flower bud has a round ball shape; mature flower bud has an elongated olive shape.
   Flower petals.—Shape: Flat, spatula-shaped Apex shape: Smooth, acute-shaped Base shape: Even obtuse Margin: Smooth.
   Flower sepal.—Shape: Delta-shaped with an acute angle at the apex. Apex shape: Triangle-shaped Margin: Smooth.
   Fragrance.—Fragrant/Moderately fragrant.
   Reproductive organs.—Fertility: Appears self-fertile Pollen amount: Abundant/Moderate amount Pollen color (general): Bright-yellow Ovary shape: Oval-shaped.
Fruit:
   Size.—Uniform.
   Height.—59.2 to 73.2 mm on average.
   Width.—67.4 to 80.5 mm on average.
   Average weight (per individual fruit).—189.8 grams.
   Shape.—Round.
   Shape (cross-section).—Round.
   Apex.—Truncated with slight dent.
   Apex cavity diameter.—N/A.
   Base cavity diameter.—7.0 to 7.1 mm.
   Base.—No neck.
   Harvesting.—Fruit can be harvested from October through December in Florida.
   Fruit stem (short stem connecting the fruit).—Length: 5.0 mm Diameter: 4.7 mm Color: RHS 196A (greyed-green) with RHS 138A (green) strip.
Rind:
   Adherence.—Adherence between albedo (mesocarp) and flesh (endocarp) is strong. The adherence is evenly distributed from base to apex.
   Thickness.—3.4 to 3.8 mm on average.
   Texture.—Smooth.
   Color.—Flavedo (epicarp): Ranges between RHS 144B (yellow-green) to RHS 144A (yellow-green) Albedo (mesocarp): RHS 155A (white).
   Stylar end.—Closed.
   Rind oil cell density.—128 oil cells/square cm.
Flesh:
   Number of segments.—Between 9 and 10 segments per fruit on average.
   Segment walls.—Medium-firm with sufficient strength to maintain integrity as separated.
   Juice.—Abundant.
   Color.—Uniformly RHS 151D (yellow-green).
   Texture.—Medium soft.
   Vesicles.—Length: Arranged from 10.3 to 21.1 mm on average Diameter (thickness): 1.5 to 2.9 mm on average.
   Eating quality.—N/A.
   Juice index.—Soluble solids (average): 9 Brix.
Seeds:
   Type.—Polyembryonic.
   Number.—Ranges from 20 to 30. Most fruit contains less than 25 seeds.
   Shape.—Seed shapes are not uniform. Normal seeds are mostly ventricose/swollen-shaped and clavate club shaped.
   Size.—Length: 12.4 to 17 mm Width: 4.8 to 6.0 mm.
   Seed coat color.—Outer Surface: RHS 155C (white) and wrinkled Inner surface: RHS 165C (greyed-orange) Cotyledon color: RHS 157B (green-white).
Resistance to Disease: ‘UFR-2’ rootstock was selected on the basis of its positive reaction to HLB disease (huanglongbing or citrus greening disease) in multiple field trials. Trees on this rootstock show a reduced frequency of infection and reduced disease symptoms once infected as compared to commercial diploid rootstocks.

What is claimed is:

1. A new and distinct citrus rootstock cultivar as illustrated and described herein.
